(19) United States
(12) Patent Application Publication
(10) Pub. No.: US 2024/0374800 A1
(43) Pub. Date: Nov. 14, 2024

(54) EXTRACORPOREAL CIRCUIT FOR DECAPNEIZATION OF ORGANIC FLUIDS

(71) Applicant: B. Braun Avitum AG, Melsungen (DE)

(72) Inventor: Giuliana Gavioli, Mirandola (IT)

(21) Appl. No.: 18/554,653

(22) PCT Filed: Apr. 6, 2022
(86) PCT No.: PCT/EP2022/059093

§ 371 Date: Oct. 10, 2023

(30) Foreign Application Priority Data
Apr. 13, 2021 (DE) 10 2021 109 242.6

(51) Int. Cl. A61M 1/16; A61M 1/34

(52) U.S. Cl. CPC A61M 1/1698; A61M 1/34; A61M 2230/208

(57) ABSTRACT

An extracorporeal circuit for the decapneization of organic fluids includes a line for draining from a patient the organic fluid to be decapneized, a line for re-infusing the patient with the decapneized organic fluid, at least one pump group of the organic fluid arranged at least on the draining line, at least one decapneizer into which the drainage line enters and from which the re-infusion line exits, and a first sensor for detecting at least one input parameter of the organic fluid to be decapneized, the first sensor being mounted between the pump group and the decapneizer.

### CROSS-REFERENCE TO RELATED APPLICATIONS

This application is the United States national stage entry of International Application No. PCT/EP2022/059093, filed on Apr. 6, 2022, and claims priority to German Application No. 10 2021 109 242.6, filed on Apr. 13, 2021. The contents of International Application No. PCT/EP2022/059093 and German Application No. 10 2021 109 242.6 are incorporated by reference herein in their entireties.

### FIELD

The disclosure relates to an extracorporeal circuit for decapneization of organic fluids, generally usable to eliminate CO2 from a patient's blood and, if required, to simultaneously filter the blood before re-infusing it into the patient.

### BACKGROUND

Machinery and methods are known to remove carbon dioxide (hereinafter CO2 for short) from the blood of a patient undergoing curative therapies and to enrich it with oxygen in cases of respiratory diseases.

Typically, this type of therapies, known as decapneization, are performed when the patient has respiratory failure due to which CO2 tends to concentrate in the blood while progressively reducing the presence of oxygen, hereinafter briefly O2.

Decapneization is a known therapy which consists, as mentioned, in the selective removal of CO2 from the patient's blood using an extracorporeal blood circuit and which involves the passage of blood flows in a decapneizer, in practice typically an oxygenator in which an O2 flow flows, typically inside hollow fibers, and blood to be decapneized, in opposite directions.

In some particular therapies, typically according to CRRT technique (Continuous Renal Replacement Therapies), a hemofilter is arranged on the extracorporeal circuit downstream of the decapneizer, to perform a microfiltration of the blood treated by the decapneizer in addition to decapneization.

The decapneization is typically used to render systems for removing CO2 less invasive.

The oxygenator or decapneizer, is supplied with O2 or medical air and works as a gas exchanger and reproduces the patient's pulmonary function.

The decapneization therapy can be performed both according to the ECMO clinical protocol, acronym for Extra Corporeal Membrane Oxygenation, and, as said above, according to the CRRT clinical protocol, acronym for Continuous Renal Replacement Therapies, in the latter case when the patient has multi-organ damage, typically a renal failure in addition to a respiratory pathology.

From patent EP 1415673 an apparatus usable in CRRT for hemofiltration treatments using a hemofiltration machine is known.

The machine is equipped with means for connecting with a patient for draining and re-infusing blood, with treatment means including a pump, with means for adding drugs and other therapeutic substances, with means for topping up a liquid into the blood and with connected filtration means, connected in cascade one to the other with respective ducts. The apparatus also has an oxygenator.

From patent EP 1698362 a machine and a unit for the treatment of blood are known. The machine includes means for the removal of CO2, and at least one outlet for a flow of blood purified of CO2. The machine also has filter means which have at least a first inlet for receiving a blood flow and at least one outlet for the outlet of a purified blood flow and a drainage channel with which, in use, a dilution liquid obtained from blood during purification is expelled. The channel is connected to the first inlet of the CO2 removal means to supply the dilution liquid to them.

From patent EP 3181164 an apparatus for the decapneization of blood is known. The apparatus includes a supply line for the blood to be treated, drained from a patient, and at least one return line suitable for transporting the treated blood to the patient. The apparatus also includes an oxygenator and at least one filtering device positioned between the power supply line and the return line. The power supply line includes a main section that can be connected on one side to the patient and connected on the other side to the oxygenator, and a by-pass section parallel to the main section, along which the filter is placed. The by-pass section includes a power supply branch connected on one side to the main section and on the other side to the filter inlet, and a return branch connected on one side to the filter outlet and on the other side to the main section. The apparatus also includes removable connection means arranged along the branches and designed to separate a first part of the branches themselves, which is connected with the filter, from a respective second part connected with the first main section; the second part can be connected to each by means of the connection means following the separation of their respective first parts.

From patent EP 3181165 an apparatus for the decapneization of the blood is known, which comprises a supply line of blood to be treated and drained from a patient and at least one line for returning the treated blood to the, at least one oxygenator and a blood filter, positioned between the supply line and the return line, first pumping means arranged to convey the blood along the supply line towards at least one of the oxygenators and the filter. The pumping means comprise at least one centrifugal pump and at least one peristaltic pump and the supply line can be connected alternatively to the centrifugal pump or to the peristaltic pump.

This state of the art has some drawbacks. One drawback is that in known extracorporeal circuits, it is not possible to determine with promptness and precision the amount of CO2 eliminated from a patient's blood in the unit of time. This data is important to allow doctors to balance the exchanges between O2 and CO2 according to patient's necessities and, therefore, to know if the amount of CO2 removed is sufficient, since, otherwise, the functional capacity of the oxygenator is progressively reduced due to the deposition of platelets and fibrin on the exchange membranes through which the exchanges occur.

In addition, the data relating to the quantities of removed CO2 provides precise indications to doctors to be able to adequately manage ventilation for the patient, preventing the onset of hypoxemia or hypercapnia.

Another drawback consists in the fact that it is desirable for doctors to be able to detect in real time, namely during the execution of the therapies, also the values of other blood parameters, e.g., the pH value or the bicarbonate HCO3 values.

As an alternative to the use of decapneization procedures, doctors for the treatment of systemic pulmonary diseases subject patients to mechanical ventilation, which, however, involves risks of complications: in particular, mechanical ventilation can lead to pulmonary barotraumas, which, in turn, may generate pneumothoraxes, emphysema, pulmonary edema, diaphragm muscle atrophy and bacterial lung infections.

### SUMMARY

The tasks and objectives of the disclosure are to eliminate or at least reduce the drawbacks of the prior art.

In other words, one purpose of the disclosure is to overcome the drawbacks noted above, by providing an extracorporeal circuit for the decapneization of organic fluids, which allows to accurately and consistently detect by sensors the CO2 values which are removed from the organic fluids, such as blood, and to prevent the arising of hypoxemia or hypercapnia.

Another purpose of the disclosure is to create an extracorporeal circuit for the decapneization of organic fluids, which allows to detect other significant parameters of organic fluids, in particular blood, also in this case in real time and with promptness.

According to one aspect of the disclosure, an extracorporeal circuit is provided for the decapneization of organic fluids, comprising a (drainage/draining) line for draining from a patient the organic fluid to be decapneized, a (re-infusion) line for re-infusing the patient with the decapneized organic fluid, at least one organic fluid pump group arranged at least on said drainage line, and at least one decapneizer into which the drainage line enters and from which the re-infusion line exits.

According to an aspect of the present disclosure, between the pump group and the decapneizer first sensor means are mounted for detecting at least one input parameter of the organic fluid to be decapneized.

Advantageous embodiments are explained below.

According to another aspect of the disclosure, on the re-infusion line second sensor means for detecting at least one output parameter of the decapneized organic fluid can be mounted downstream of the decapneizer.

In a beneficial embodiment, the extracorporeal circuit may further comprise a haemofilter, wherein the first sensor means and the second sensor means can be arranged upstream of the haemofilter. In other words, according to the beneficial embodiment, the haemofilter can be arranged downstream of the first and second sensor means, such that the organic fluid can flow within the drainage line through the pump group, the first sensor means, the decapneizer, the second sensor means and the haemofilter. Further, it is preferable, that the haemofilter is connected to the re-infusion line, such that, after passing through the haemofilter, the organic fluid may enter the re-infusion line. By arranging the first and second sensor means upstream of the haemofilter, the influence of haemofiltration on the determination of the parameters, in particular CO2 values, detected by the sensor means can be avoided.

According to a further preferred embodiment of the disclosure, the first sensor means and the second sensor means can be mounted at selected distances with respect to the decapneizer. Preferably the first sensor means can be mounted at a first distance with respect to the decapneizer, and the second sensor means can be mounted at a second distance with respect to the decapneizer, and wherein the second distance can be greater than or equal to, preferably greater than, the first distance. Particularly preferable the first distance can vary in a range between 1 to 2 cm, and the second distance can vary in a range between 1 to 10 cm. In other words, the distances can vary in a range of 1 to 10 cm, wherein a distance between the first sensor means and the decapneizer can vary between 1 and 2 cm, and a distance between the second sensor means and the decapneizer can vary between 1 and 10 cm. When arranging the first and second sensor means at said distances, possible falsification of the parameters detected by the sensor means due to the flow conditions within the drainage line and due to gas diffusion inside the decapneizer can be reduced. In other words, the first sensor means are arranged as close as possible to the decapneizer, but at a distance sufficient enough to have the detected parameter not contaminated/influenced by the possible gas diffusion inside the decapneizer or the flow within the drainage line. In other words, according to a preferable embodiment, the first sensor means may be mounted closer to the decapneizer than the second sensor means, since the second sensor means are less keen to be influenced by gas diffusion inside the decapneizer. Nevertheless, both of the first and second sensor means have to be mounted to the decapneizer at a minimum distance of about 1 cm.

Preferably, the first sensor means and the second sensor means can be mounted directly on the decapneizer, in such a way as to be monolithic with it, such that distances between the decapenizer and the first and second sensor means, respectively, can be maintained at a predetermined value.

Further, the above-mentioned input and output parameter can be chosen in a group consisting of: CO2 partial pressure (PiCO2), CO2 concentration (TiCO2), pH value, bicarbonate (HCO3) concentration.

Preferably, the input and output parameter can be normalized per unit of blood, for example per 1 liter of blood.

According to an embodiment of the disclosure, a decapneizer can be provided, which includes a containing body in which a flow of oxygen is intended to flow, a first inlet of oxygen and a first outlet of carbon dioxide, a second inlet of a drainage line for draining from a patient a fluid to be decapneized, a second outlet of a re-infusion line for re-infusing the patient with decapneized fluid. According to this embodiment first sensor means are associated with the containing body at the second inlet.

The extracorporeal circuit and the decapneizer of the present disclosure achieve the following advantages:
To decapneize organic fluids, in particular blood, maintaining a constant control, in real time, of the removed CO2 values;
To avoid the use of mechanical ventilation of the patients;
To monitor O2 and CO2 values and to balance their ratio in real time and according to specific necessities of each patient and of any kind of therapy to be performed.

### BRIEF DESCRIPTION OF THE DRAWINGS

Further characteristics and advantages of the disclosure will become more evident from the detailed description of preferred, but not exclusive, embodiments of an extracorporeal circuit for the decapneization of organic fluids, shown by way of a non-limiting example in the attached drawings wherein:

FIG. 1 is a schematic view of an extracorporeal circuit for the decapneization of organic fluids, according to an isolated technique;

FIG. 2 is a schematic view of an extracorporeal circuit for the decapneization of organic fluids, according to the CRRT technique; and

FIG. 3 is a schematic view in an enlarged scale of a possible alternative version of a decapneizer, which is a component of the extracorporeal circuit for decapneization of organic fluids according to the disclosure.

### DETAILED DESCRIPTION

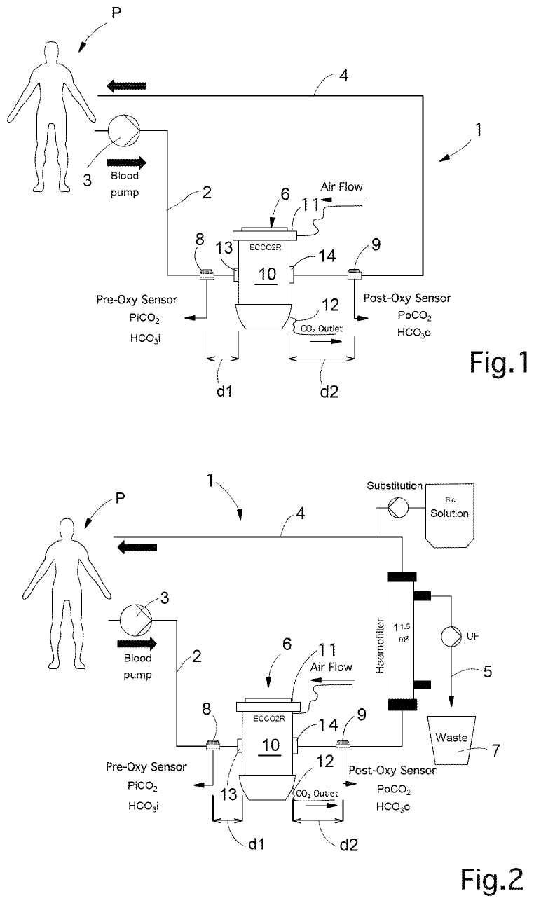

With reference to the drawings, P indicates a patient treated with decapneization of the isolated type (FIG. 1) and of the CRRT type (FIG. 2), whilst the extracorporeal circuit is indicated overall with reference number 1. In both cases, an organic fluid is drained from a patient in a known way, in the specific and exemplary case the blood, through a drainage line 2 on which at least one pump or pump unit 3 for suction and thrust acts. The decapneized blood is re-infused to patient P, through a re-infusion line 4 and with the thrust action of the pump or pump group 3.

The generic term “line” means a duct with a caliber able to be selected according to the desired flow rates of organic fluids, preferably made of biocompatible and flexible plastic material, inside which a flow of blood or other organic fluid can flow. At the ends, the duct is equipped with known connections for devices for accessing the vessels of the patient's blood circuit, for example catheters.

As can be seen in the drawings, in both cases, a decapneizer 6 is mounted on line 2, inside which the CO2-O2 exchange takes place in a known way respectively from, and in, the blood, typically in an osmotic manner through a membrane or bundles of gas-permeable but hydrophobic hollow fibers, which are housed inside the decapneizer 6, interposed between the blood flows and the O2 flows that flow inside them. Typically, through the membrane or the hollow fibers the exchange takes place due to the difference in partial pressures between O2 and CO2 and thanks to which O2 enters the blood, while CO2 is eliminated and collected in a container 7 through a discharge line 5 provided for this function.

In general, the decapneizer 6 comprises a body 10 wherein a flow of oxygen is intended to flow. The body 10 has at least a first inlet 11 of oxygen and a first outlet 12 of carbon dioxide and a second inlet 13 of a line 2 for draining from a patient P a fluid to be decapneized, a second outlet 14 of a line for re-infusing 4 the patient P with decapneized fluid.

As can be seen in both FIGS. 1 and 2, a first sensor 8 and a second sensor 9 placed at respective distances d1 and d2 with respect to the decapneizer 6 are mounted respectively upstream and downstream of the decapneizer 6.

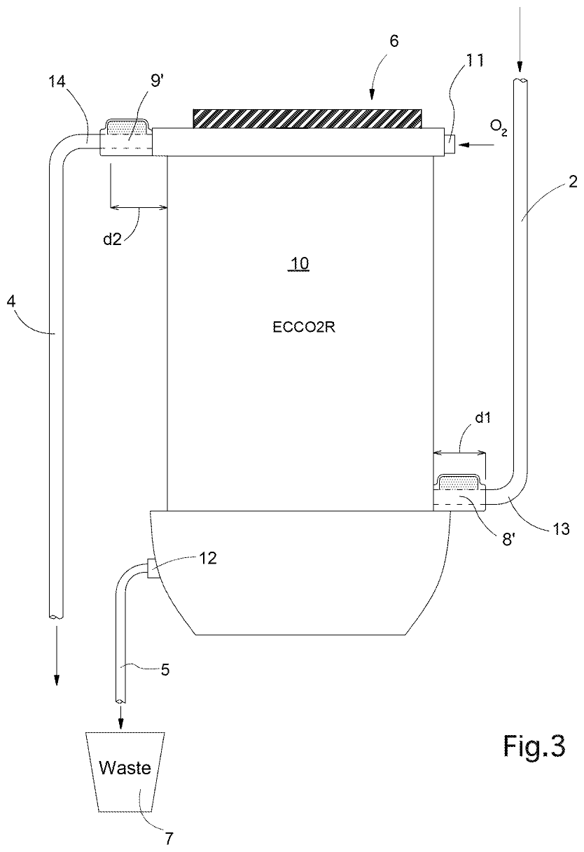

In FIG. 3, which shows a particular version of the decapneizer 6, the first sensor 8′ and the second sensor 9′ are mounted directly on the body of the latter, in such a way as to be monolithic with it and to maintain at the same time the distances d1 and d2. Preferably, the values of the distances d1 and d2 can vary in a range comprised between 1 to 10 cm. More specifically distance d1 can vary between 1 to 2 cm while distance d2 can vary between 1 and 10 cm.

The first sensors 8, 8′ are configured to detect an input parameter of the blood entering the decapneizer 6, whilst the second sensors 9, 9′ are configured to detect an output parameter of the decapneized blood. The expression “input parameter” and “output parameter” mean a parameter of the blood respectively before and after the decapneization process has occurred.

In the embodiment of the extracorporeal circuit 1 shown in FIG. 1, which shows an embodiment intended for isolated decapneization, the two sensors 8 and 9 are placed at the predetermined distances indicated with d1 and d2, such as to prevent the flows at entry and at exit from the decapneizer 6 from being affected by dynamic influences which can affect the values of the parameters detected by the sensors 8 and 9, thus providing incorrect values.

The person skilled in the art understands that the two distances d1 and d2 can be both the same or different from one another, depending on the structure of the extracorporeal circuit 1 and on the kind of organic fluid to be subjected to decapneization. The shape of the decapneizer body can also affect the values of the distances d1 and d2 when sensors 8′ and 9′ are an integral part of it.

The operation of the disclosure is substantially similar to that of a conventional extracorporeal circuit, with the difference that the presence of two sensors 8 and 9 (or 8′ and 9′) allows to detect one or more characteristic parameters of the decapneized organic fluid in real time, in the exemplary case, of the blood while it flows inside the extracorporeal circuit 1, providing doctors with timely and precise values of the analyzed parameters, so that doctors can customize respiratory therapies to the needs and stable or unstable conditions of each patient.

Sensors 8 and 9 may be sensors for individually detecting the percentages of CO2, or the pH value, or even hydrogen carbonate (HCO3) values (bicarbonate), or they may also be multi-purpose sensors capable of simultaneously detecting multiple parameters to be monitored. Furthermore, sensors 8 and 9 or 8′ and 9′ may be the same or even different.

In practice it has been found that the disclosure achieves the intended purposes. The disclosure as conceived is susceptible of modifications and variations, all falling within the inventive concept. Furthermore, all details can be replaced with other technically equivalent elements. Further, the materials used as well as the shapes and sizes may be any, depending on requirements.

1. An extracorporeal circuit for decapneization of an organic fluid, the extracorporeal circuit comprising:
a drainage line for draining from a patient the organic fluid to be decapneized;
a re-infusion line for re-infusing the patient with the organic fluid after decapneization;
at least one organic fluid pump group arranged at least on said drainage line;
at least one decapneizer into which the drainage line enters and from which the re-infusion line exits; and
a first sensor mounted between the at least one organic pump group and the decapneizer for detecting at least one input parameter of the organic fluid.

2. The extracorporeal circuit according to claim 1, further comprising a second sensor on the re-infusion line for detecting at least one output parameter of the organic fluid, the second sensor mounted downstream of the decapneizer.

3. The extracorporeal circuit according to claim 1, further comprising a haemofilter, wherein the first sensor and the second sensor are arranged upstream of the heamofilter.

4. The extracorporeal circuit according to claim 3, wherein the first sensor and the second sensor are mounted at selected distances from the decapneizer.

5. The extracorporeal circuit according to claim 4, wherein the first sensor is mounted at a first distance from the decapneizer, and the second sensor is mounted at a second distance from the decapneizer, and wherein the second distance is greater than or equal to the first distance.

6. The extracorporeal circuit according to claim 5, wherein the first distance varies in a range between 1 to 2 cm, and the second distance varies in a range between 1 to 10 cm.

7. The extracorporeal circuit according to claim 1, wherein said first sensor is selected from CO2 pressure sensors, pH sensors, and HCO3 bicarbonate sensors.

8. The extracorporeal circuit according to claim 2, wherein said second sensor is selected from CO2 pressure sensors, pH sensors, and HCO3 bicarbonate sensors.

9. The extracorporeal circuit according to claim 2, wherein said first sensor and second sensor are the same or different from each other.

10. The extracorporeal circuit according to claim 2, wherein said at least one input parameter and said at least one output parameter are chosen from the group consisting of: CO2 partial pressure (PiCO2), CO2 concentration (TiCO2), pH value, and bicarbonate concentration.

11. A decapneizer comprising:
a containing body in which a flow of oxygen is intended to flow;
a first inlet of oxygen and a first outlet of carbon dioxide;
a second inlet of a line for draining from a patient a fluid to be decapneized;
a second outlet of a line for re-infusing the patient with decapneized fluid; and
a first sensor associated with said containing body in correspondence with said second inlet.

12. The decapneizer according to claim 11, further comprising a second sensor associated with said containing body in correspondence with said second outlet.
